(19) United States
(12) Patent Application Publication
(10) Pub. No.: US 2024/0180878 A1
(43) Pub. Date: Jun. 6, 2024

(54) AMIDE DERIVATIVES OF BUTYRIC ACID FOR USE IN THE TREATMENT OR PREVENTION OF SARS-COV-2 INFECTION

(71) Applicant: RHEA INNOVATIONS S.R.L., Milano (IT)

(72) Inventor: Carmine SPERA, Napoli (IT)

(21) Appl. No.: 18/554,940

(22) PCT Filed: Apr. 13, 2022
(86) PCT No.: PCT/EP2022/059828

§ 371 Date: Oct. 11, 2023

(30) Foreign Application Priority Data
Apr. 15, 2021 (IT) 102021000009497

(51) Int. Cl. A61K 31/4174; A61K 31/165; A61K 45/06; A61P 31/14

(52) U.S. Cl. CPC A61K 31/4174; A61K 31/165; A61K 45/06; A61P 31/14

(57) ABSTRACT

Methods for preventing or reducing SARS-CoV-2 entry in a subject's cells and for preventing or reducing an inflammatory cascade induced by a SARS-CoV-2 infection in a subject that involve administering to the subject of an effective amount of a composition including an amide derivative of butyric acid or a mixture of amide derivatives of butyric acid are provided. Preferably, the amide derivative of butyric acid is N-(1-carbamoyl-2-phenyl-ethyl) butyramide (FBA).

### FIELD OF THE INVENTION

The invention relates to amide derivatives of butyric acid for use in the prevention or treatment of SARS-CoV-2 infection.

### BACKGROUND ART

The COVID-19 pandemic, also known as the coronavirus pandemic, is an ongoing global pandemic of coronavirus disease 2019 (COVID-19) caused by severe acute respiratory syndrome coronavirus 2 (SARS-CoV-2)(1). This virus mainly affects the respiratory system and the gastrointestinal tract. ACE2 (angiotensin-converting enzyme-2) and TMPRSS2 (transmembrane protease serine-2) are key molecules in SARS-Cov-2 infection, and are expressed in the aforementioned tissues(2). Under siege by SARS-CoV-2, host defenses launch a counterattack releasing massive amounts of cytokines, resulting in a “cytokine storm” (3). The “cytokine storm” attempts to destroy the infecting virus but, in the process, collateral damages occur in several human tissues, mainly in the lung and in the gastrointestinal tract(4). The inflammation accompanying SARS-CoV-2 infection results in high blood levels of several proinflammatory cytokines(5). Elevated proinflammatory cytokines in severe Covid-19 patients is a predictor of higher mortality rates(6). Some successes have been reported upon treatment of Covid-19 patients with anti-inflammatory drugs(5).

However, there is still a large need for an effective therapy against SARS-Cov-2 infection.

Butyrate is a short chain fatty acid (SCFA) produced by the gut microbiome with a pivotal role for human health(6). COVID-19 patients show marked alteration in gut microbiome structure (dysbiosis)(8). These alterations include a decrease in carbohydrate fermentation and a dramatic decline in the production of SCFA, most notably butyrate(8).

Butyrate is a well-characterized short chain fatty acid and is known to act as a key modulator for defense against bacterial (9) and viral infections (10). Deficiency of butyrate is known to negatively impact immune defense(7). Butyrate is also able to exert a potent anti-inflammatory action in human tissues(11,12).

However, the use of butyrate in human nutrition is very limited due to its negative organoleptic profile, which is characterized by an extremely offensive odor and taste(13).

### BRIEF DESCRIPTION OF THE INVENTION

The inventor has surprisingly found that N-(1-carbamoyl-2-phenyl-ethyl) butyramide (FBA), a known butyric acid releaser that shows physicochemical characteristics suitable for oral administration being entirely free of the unpleasant organoleptic properties that characterize butyrate(13), is able to counteract several aspects of SARS-CoV-2 infection. The inventor has found that FBA is able to downregulate the expression of molecules that are necessary for virus entry and replication in human cells, and of pro-inflammatory cytokines. Accordingly, FBA is effective in preventing or reducing the attachment of SARS-CoV-2 to human cells, its replication in human cells and the inflammatory cascade induced by this pathogen.

Without being bound to any theory, it is believed that supplementation of Covid-19 patients with FBA may exert a favorable effect by releasing butyrate into the gastrointestinal tract, which contains one of the highest concentrations of SARS-CoV-2 receptors, and increasing butyrate absorption for systemic distribution.

It is plausible that the aforementioned favorable effects shall also extend to individuals not yet infected with SARS-CoV-2 but exposed to this pathogen, thereby preventing or reducing the occurrence of SARS-CoV-2 infection and/or its harmful effects on human body.

It is also envisaged that the aforementioned favorable effects shall also extend to other known amide derivatives of butyric acid that show the same physicochemical, organoleptic and pharmacokinetic properties as FBA. Such amide derivatives of butyric acid and their properties are disclosed in International patent application WO2009130735A1 to Rhea Innovations S.r.l. They include:
N-(1-carbamoyl-2-phenyl-ethyl)butyramide (FBA);
N-(1-butyroyl-carbamoyl-2-phenyl-ethyl)butyramide;
5-benzyl-2-propyl-1H-imidazol-4(5H)-one;
N-(1-oxo-3-phenyl-1-(piperidin-1-yl)propan-2-yl)butyramide;
N-(1-oxo-3-phenyl-1-(pyrrolidin-1-yl)propan-2-yl)butyramide;
N-(1-(methylcarbamoyl)-2-phenylethyl)butyramide;
N-(1-(ethylcarbamoyl)-2-phenylethyl)butyramide;
N-(1-(propylcarbamoyl)-2-phenylethyl)butyramide;
N-(1-(butylcarbamoyl)-2-phenylethyl)butyramide;
N-(1-(pentylcarbamoyl)-2-phenylethyl)butyramide;
N-(1-carbamoyl-2-phenylethyl)-N-methylbutyramide;
N-(1-carbamoyl-2-phenylethyl)-N-ethylbutyramide; and
N-(1-carbamoyl-2-phenylethyl)-N-propylbutyramide;
and pharmaceutically acceptable salts, diastereoisomes and enantiomers thereof.

Further features and advantages of the invention will become apparent from the following detailed description and experimental examples.

### DETAILED DESCRIPTION OF THE INVENTION

In a first aspect, the invention relates to a composition comprising an amide derivative of butyric acid or a mixture of amide derivatives of butyric acid as defined in appended claim 1, for use in the treatment or prevention of SARS-CoV-2 infection.

The preferred amide derivative of butyric acid for use according to the invention is N-(1-carbamoyl-2-phenyl-ethyl) butyramide (FBA), but further individual derivatives and mixtures of derivatives are identified in the appended claims, which form an integral part of the description.

The composition of the invention is effective in treating or preventing infection by Sars-Cov-2 and, consequently, the symptoms and disorders related to SARS-CoV-2 infection, which may include respiratory, gastrointestinal, hepatic, neurological, and systemic signs and symptoms, such as fever, cough, chills, muscle aches, headache, abdominal pain, vomiting, diarrhea and breathing difficulties.

In the composition of the invention, the amide derivative of butyric acid or the mixture of amide derivatives of butyric acid are optionally in combination with one or more further active compounds or substances known to be beneficial against SARS-CoV-2 infection.

Active compounds or substances which are already known in the prior art to exert a beneficial effect against SARS-CoV-2 infection and which may be included in the composition of the invention include synthetic and/or natural active compounds or substances, as well as food ingredients. Illustrative examples of such further active compounds and substances are flavonoids (such as quercetin, kaempferol, rutin or rutoside, naringenin, apigenin, silymarin, hesperidin, luteolin, cyanidin, gallate of epigallo-catechin, genistein), vitamins (such as vitamin A, vitamin B2, vitamin B6, Folate, vitamin B12, vitamin C, vitamin D), minerals (such as zinc, copper, iron, selenium), omega-3-polyunsatured fatty acids (such as eicosapentaenoic acid and docosahexaenoic acid), curcumin, berberine, lactoferrin, prebiotics (such as inulin, fructooligosaccharides, galactooligosaccharides, resistant starch, polydextrose, human milk oligosaccharides), probiotics, radical scavengers (such as ellagic acid and polyphenols)(14-24).

In Table 1 are reported the daily dose ranges suitable for the preparation of food supplements according to the invention, containing FBA as the amide derivative of butyric acid in combination with the aforementioned further active ingredients.

TABLE 1

| Active compound | Daily dose range | |
|---|---|---|
| FBA | 2-4 | g |
| Quercetin | 100-200 | mg |
| Vit. A | 250-1200 | μg |
| Vit. E | 5-60 | mg |
| Vit. D | 10-35 | μg |
| Folate | 80-400 | μg |
| Vit. B6 | 1.6-10 | mg |
| Vit. B12 | 1.5-1000 | μg |
| Vit. C | 20-1000 | mg |
| Curcumin | 400-500 | mg |
| Berberine | 10-400 | mg |
| Genistein | 10-80 | mg |
| Lactoferrin | 50-200 | mg |
| Omega-3-PUFAs | 5 g (EPA + DHA) | |
| Prebiotics: | | |
| inulin | 5-12 | g |
| fructooligosaccharides(FOSs) | 2-10 | g |
| galactooligosaccharides(GOSs) | 2-17 | g |
| resistant starch | 5-10 | g |
| polydextrose | 10-50 | g |
| human milk oligosaccharides(HMOs) | 0.15-0.5 | g |
| Probiotics | 10⁷-10¹⁴ | CFU |
| Radical scavengers | | |
| Ellagic acid | 10-100 | mg |
| Polyphenols(hydroxytyrosol) | 1-5 | mg |
| Zinc | 2-10 | mg |
| Iron | 10-30 | mg |
| Copper | 0.4-2 | mg |
| Selenium | 10-100 | μg |

Conveniently, the composition for use according to the invention is formulated as an enteral, parenteral, topical or oral preparation.

As mentioned, N-(1-carbamoyl-2-phenyl-ethyl) butyramide (FBA) as well as the other amide derivatives of butyric acid disclosed in WO2009130735A1 are particularly suitable for oral administration, as they are entirely free of the unpleasant organoleptic properties that characterize butyrate. Accordingly, a preferred composition for use according to the invention is formulated as an oral composition.

Examples of oral compositions falling within the scope of the invention are food products, food supplements, drinks and beverages, as well as oral pharmaceutical forms, such as tablets, sachets, pills, capsules, syrups.

In the case of food supplements or pharmaceutical forms, the composition for use according to the invention may also include pharmaceutically or dietarily acceptable vehicles and/or excipients, the selection of which is largely within the ability of the person skilled in the art.

Examples of food products within the scope of the invention include energy bars, candys, chewing gum, bubble gum, lollipop, enteral formulas, artificial nutrition products, food for special dietary uses, foods for special medical purposes.

The composition for use according to the invention may also be administered in combination with pharmaceutical active ingredients known to be effective in the treatment of SARS-Cov-2 infection or symptoms and disorders related thereto, including inter alia antibiotics (for example, macrolide or lincosamide antibiotics, such as erythromycin, azithromycin or clindamycin), antivirals (such as zidovudine, stavudine, indinavir, saquinavir, efavirenz or ribavirin), Non-Steroidal Anti-Inflammatory Drugs (NSAIDs) (such as cecloxibe, diclofenac, flurbiprofen, ibuprofen, ketoprofen, meloxicam, naproxen or acetylsalicylic acid), peroxisome proliferator-activated receptor alpha (PPARα) agonists, analgesics (such as acetaminophen), Steroidal Anti-Inflammatory Drugs (SAIDs) (such as budesonide, beclometasone, prednisone or hydrocortisone), protease inhibitors (such as Darunavir/cobicistat or atazanavir/cobicistat), anti TNFα biopharmaceutical drugs (such as infliximab or etanercept), anti-cytochine biopharmaceutical drugs (such as anakinra or tocilizumab), mucolytics (such as carbocysteine, ambroxol or acetylcysteine). In this embodiment, it is preferred that the aforementioned pharmaceutical active ingredients are not physically mixed with the composition for use according to the invention but are provided as a combined preparation for simultaneous, separate or sequential use in the therapeutic treatment or prevention of SARS-CoV-2 infection.

Accordingly, a further aspect of the invention is a kit-of-parts comprising a composition comprising at least one amide derivative of butyric acid as defined above and at least one pharmaceutical active ingredient selected from the group consisting of antibiotics, antivirals, NSAIDs, PPARα agonists, analgesics, SAIDs, protease inhibitors, mucolytics, anti-TNFα biopharmaceutical drugs and anti-cytokine biopharmaceutical drugs, as a combined preparation for simultaneous, separate or sequential use in the treatment or prevention of SARS-CoV-2 infection.

The following examples demonstrate the beneficial effects of FBA against SARS-Cov-2. The examples are provided for illustrative purposes only and are not intended to limit the scope of the invention as defined in the appended claims.

The examples make reference to the appended drawings, in which:

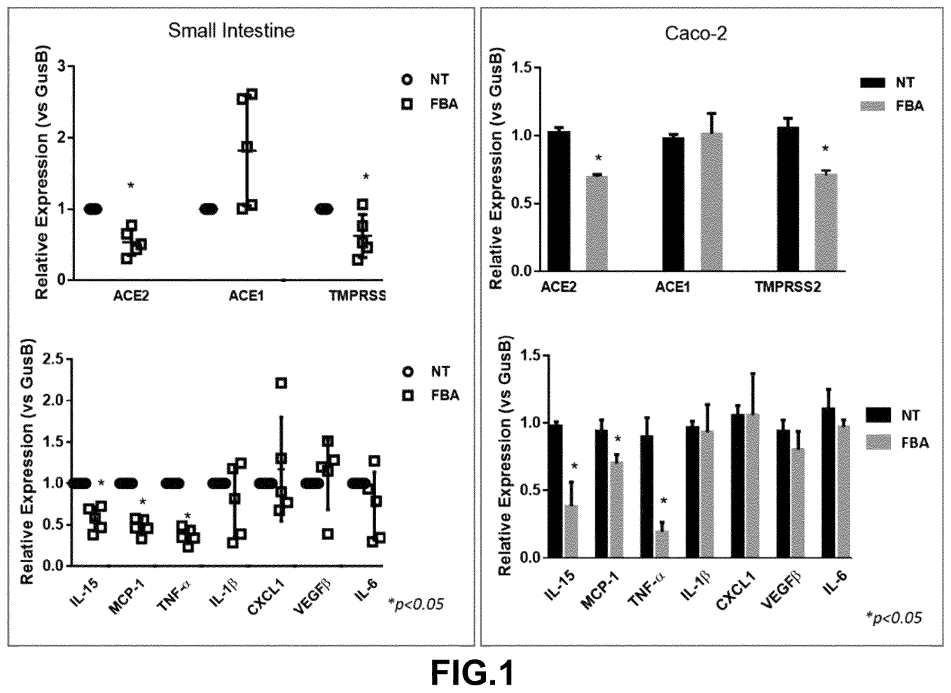

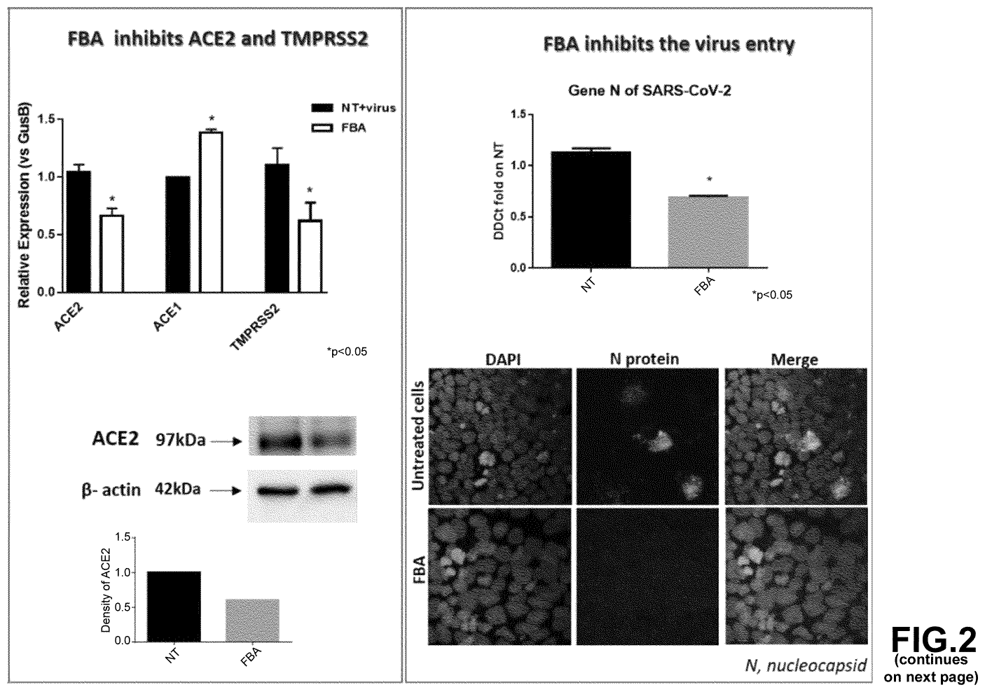

FIG. 1 shows the effect of FBA on the main cellular compounds involved in SARS-CoV-2 infection and on cytokines expression in human intestinal tissue;

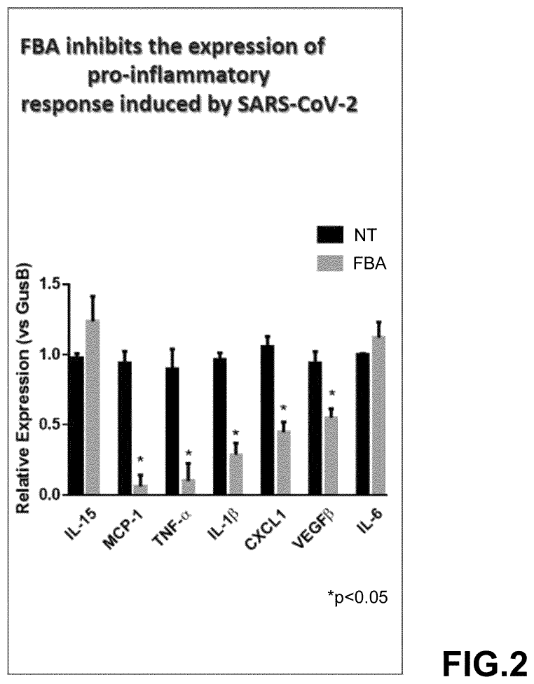

FIG. 2 shows the effect of FBA on SARS-CoV-2 infection in human cells.

### EXAMPLES

### Organ Culture Studies

Based on the evidence that the intestinal tract is a main entry site for SARS-CoV-2 (2), in a first set of experiments the inventor investigated the direct effect of FBA on main players of SARS-CoV-2 infection (angiotensin-converting enzyme 2, ACE2; angiotensin-converting enzyme 1, ACE1; transmembrane serine protease-2, TMPRSS2)(2), and on pro-inflammatory cytokines response in human small intestine tissue. Small intestinal biopsies were collected by esophago-gastro-duodenoscopy from 5 healthy control subjects (3 men and 2 female, age range 11-34 years). Small intestinal biopsy culture was performed in RPMI-1640 medium (Sigma-Aldrich, Germany) without L-glutamine and supplemented with 10% fetal calf serum and antibiotic/antimycotic mixture (Gibco Invitrogen). Each sample was divided in two fragments for different conditions: medium alone (as negative control) and treatment with FBA (2 mM). The fragments were placed on a stainless steel mesh positioned over the central well of an organ culture dish with the villous surface of the specimens uppermost. They were cultured for 24 h. Then, the specimens were immediately placed in RNAlater (Thermo Fisher Scientific, Waltham, MA, USA) and stored at −80° C. until analysis for RNA extraction. Human small intestine biopsy samples collected form 5 healthy subjects was incubated with FBA at different doses and times for RNA extraction. Total RNA was extracted with the TRIzol reagent (Invitrogen, Thermo Scientific, Waltham, MA, USA). All samples were quantified using theNanoDrop 2000c spectrophotometer (Thermo Scientific) and RNA quality and integrity were assessed with the Experion RNA Standard Sense kit (Bio-Rad, Hercules, CA, USA). cDNA was synthesized with random primers using the SensiFASTcDNA Synthesis Kit (Bioline) on theCFX96 RealTime System instrument (Bio-Rad, Hercules, CA, USA). Quantitative real-time PCR (qRT-PCR) analysis was performed using Taqman Gene Expression Master Mix (Applied Biosystems; Vilnius, Lithuania) to evaluate the gene expression of ACE2, ACE1, TMPRSS2, IL-15, MCP-1, TNF-α, IL1β, CXCL1, VEGFβ, and IL-6, using specific TaqMAn probes. The TaqMan probes for these genes were inventoried and tested by Applied Biosystems manufacturing facility (QC). Amplification conditions were initial steps at 50° C. for 2 min and 95° C. for 10 min, followed by 40 cycles of 95° C. for 15 s and 60° C. for 1 min in an Applied Biosystems ABI PRISM 7900HT Sequence Detection system. Data analysis was performed using the comparative threshold cycle (CT) method and expressed as 2\hat{ }-delta CT. Gene expression was normalized against the expression of the reference gene glyceraldeide-3-phosphate dehydrogenase (GAPDH).

The inventor found that the expression of ACE2 and TMPRSS2, which are the cellular mediators that facilitate SARS-CoV-2 entry into host cells, were both significantly decreased by the incubation with 2 mM FBA for 24 h. Furthermore, the stimulation with 2 mM FBA elicited a significant reduction of the expression of the following pro-inflammatory cytokines: interleukin (IL)-15, monocyte chemoattractant protein-1 (MCP-1) and tumor necrosis factor-alpha (TNF-α) (FIG. 1).

### Human Enterocytes Cell Line Studies

Then, another set of experiments was performed on human enterocytes cell line (Caco-2 cells), after 15 day of differentiation, to demonstrate the reproducibility of these effects also in this experimental model. Caco-2 cells were purchased from the American Type Culture Collection (ATCC; Teddington, UK). Cells were cultured in high glucose Dulbecco's modified Eagle medium (DMEM; Gibco, Berlin, Germany) with 10% Fetal bovine serum (Gibco, Paisley, UK), 1% Non-Essential amino acids (Gibco, Paisley, UK), 1% (v/v) antibiotics (10.000 U/mL Penicillin and 10 mg/mL Streptomycin) (EuroClone, Pero, Italy), and 2 mM L-Glutamine (Gibco, Paisley, UK). Caco-2 cells were kept at 37° C. in a 5% CO₂ and 95% air humidified atmosphere. The culture medium was changed every 2 days. After 15 day of differentiation, Caco-2 cells were stimulated with FBA (at different doses and times of incubation) or with medium alone for 24 h. Then, the cells were immediately placed in RNAlater (Thermo Fisher Scientific, Waltham, MA, USA) and stored at −80° C. until analysis for RNA extraction. All experiments were performed in triplicate and were repeated three times. RNA samples were extracted, processed and analyzed as described above.

Similar results as those illustrated above were obtained: 2 mM FBA was the maximal effective dose to significantly reduce ACE2, TMPRSS2 and IL-15, MCP-1 and TNF-α expression in human enterocytes after 24 h of incubation (FIG. 1).

### Human Epithelial Respiratory Cells Culture Studies

To confirm these results also in respiratory epithelial cells, the human respiratory epithelial cell line Calu3 was used. Calu-3 cells (American Type Culture Collection; Rockville, MD) were cultured at 37° C. under 5% CO₂ in complete EMEM medium supplemented with 20% FBS, and 1% penicillin-streptomycin. Cells were seeded at an initial concentration of 5×10⁵ cells/mL and medium was changed every 3 days. Cells were stimulated with FBA (at different doses and times of incubation) or with medium alone for 24 h. Then, the cells were immediately placed in RNAlater (Thermo Fisher Scientific, Waltham, MA, USA) and stored at −80° C. until analysis for RNA extraction. All experiments were performed in triplicate and were repeated three times. RNA samples were extracted, processed and analyzed as described above.

Also in this experimental model, the beneficial effects elicited by FBA were confirmed. After 24 h of incubation, 2 mM FBA was able to downregulate the expression of ACE2, TMPRSS2 and IL-15, MCP-1 and TNF-α.

### SARS-CoV-2 Infection Model

In a second set of experiments, the direct effect of FBA on ACE2, ACE1 and TMPRSS2 and on pro-inflammatory cytokines response in an in vitro model of SARS-CoV-2 infection was investigated.

The SARS-CoV-2 infection was performed on Caco-2, at 15 days post-confluence, using SARS-CoV-2 wild-type strain. Before the infection, cells were incubated with 2 mM of FBA for 24 h at 37° C. 1 MOI of SARS-CoV-2 was added to the Caco-2 monolayer for 72 h. Then, SARS-CoV-2 inoculum was removed, the cells were washed three times with PBS 1× and were harvested for RNA, proteins extraction and for fluorescence microscopy analysis.

RNA samples were extracted, processed and analyzed as described above. Western blotting was performed on the total protein extracts of infected Caco-2 cells pretreated with FBA. For the total protein fraction, the harvested cells were washed in cold phosphate-buffered saline (PBS) and lysed in protein lysis buffer (RIPA). Protein concentrations in cell extracts were determined using the Bradford assay (BioRad, Milan, Italy). Thirty microgram total lysates were loaded onto 10% SDS-PAGE and then transferred to nitrocellulose membranes (ImmobilonR-Transfer Membrane, Tullagreen, Carrigtwohill, Co). The membranes were blocked with 5% non-fat milk in PBS, pH 7.6, 0.2% Tween 20 (PanReac AppliChem) and probed overnight at 4° C. with the specific primary antibodies for ACE2 (1:2000; Abcam). After washing in PBS, pH 7.6, 0.2% Tween 20, the membranes were incubated with a horseradish peroxidase-conjugated goat anti-rabbit antibody (1:2000; Abcam). The immunoblots were visualized using ECL detection kits, with enhanced chemiluminescence (Pierce, Rockford, IL, USA). A mouse β-actin antibody (1:5000; Elabscience) was used as the control for equal loading of total lysates.

The SARS-CoV-2 Nucleocapsid (N) protein was labeled in infected Caco-2 cells pretreated with FBA. Briefly, Caco-2 cell monolayers were washed and fixed with absolute ice-cold methanol for 10 min at room temperature. Cover slips were washed twice with PBS, then the cells were permeabilized with Triton X-100 (PanReac AppliChem) in PBS for 10 min. After washing, cells were blocked for 1 hour using 1% BSA in PBS/Tween 20 (PanReac AppliChem) and then incubated overnight at 4° C. with specific primary antibody for rabbit polyclonal anti-SARS-CoV-2 Nucleocapsid (N) protein (Novus Biologicals 100-56576, 0.5 mg/ml). Nuclei were stained with 4′6-Diamidino-2-phenylindole dihydrochloride (DAPI) (Life Technologies, Willow Creek Road, Eugene, Oregon). Finally, cells were mounted with antifading mowiol and viewed using an inverted fluorescence microscope.

Western blotting was performed on the total protein extracts of infected Caco-2 cells pretreated with FBA. For the total protein fraction, the harvested cells were washed in cold phosphate-buffered saline (PBS) and lysed in protein lysis buffer (RIPA). Protein concentrations in cell extracts were determined using the Bradford assay (BioRad, Milan, Italy). Thirty microgram total lysates were loaded onto 10% SDS-PAGE and then transferred to nitrocellulose membranes (ImmobilonR-Transfer Membrane, Tullagreen, Carrigtwohill, Co). The membranes were blocked with 5% non-fat milk in PBS, pH 7.6, 0.2% Tween 20 (PanReac AppliChem) and probed overnight at 4° C. with the specific primary antibodies for ACE2 (1:2000; Abcam). After washing in PBS, pH 7.6, 0.2% Tween 20, the membranes were incubated with a horseradish peroxidase-conjugated goat anti-rabbit antibody (1:2000; Abcam). The immunoblots were visualized using ECL detection kits, with enhanced chemiluminescence (Pierce, Rockford, IL, USA). A mouse 3-actin antibody (1:5000; Elabscience) was used as the control for equal loading of total lysates.

Surprisingly, the inventor observed that FBA was able to inhibit the virus entry, the ACE2 and TMPRSS2 expression and the expression of pro-inflammatory cytokines (MCP-1, TNF-α, IL-1β, CXCL1 and VEGFβ) in human enterocytes exposed to wild-type SARS-CoV-2 (FIG. 2). Similar results were obtained in B.1.1.7 SARS-CoV-2 variant-infected cells.

### Statistical Analysis

The Kolmogorov-Smirnov test was used to determine whether variables were normally distributed. Descriptive statistics were reported as means and standard deviations (SDs) for continuous variables. To evaluate the differences among continuous variables, the independent sample t-test was performed. The level of significance for all statistical tests was two-sided, p<0.05. All data were collected in a dedicated database and analyzed by a statistician using GraphPad Prism 7 (La Jolla, CA, USA).

### REFERENCES

1. Atzrodt C L, et al. A Guide to COVID-19: a global pandemic caused by the novel coronavirus SARS-CoV-2. FEBS J. 2020; 287:3633-3650
2. Hoffmann M, et al. SARS-CoV-2 cell entry depends on ACE2 and TMPRSS2 and is blocked by a clinically proven protease inhibitor. Cell. 2020; 181:271-280
3. Mehta P, et al. COVID-19: consider cytokine storm syndromes and immunosuppression. Lancet. 2020; 395:1033-1034
4. Liao M, et al. The landscape of lung bronchoalveolar immune cells in COVID-19 revealed by single-cell RNA sequencing. MedRxiv. 2020; 10.1101/2020.02.23.20026690
5. Zhang W, et al. The use of anti-inflammatory drugs in the treatment of people with severe coronavirus disease 2019 (COVID-19): the experience of clinical immunologists from China. Clin Immunol. 2020; 214:108393.
6. Zhou F, et al. Clinical course and risk factors for mortality of adult inpatients with COVID-19 in Wuhan, China: a retrospective cohort study. Lancet. 2020; 395:1054-62
7. Zheng, D, et al. Interaction between microbiota and immunity in health and disease. Cell Res 2020; 30:492-506
8. Shinde T, et al. Microbiota modulating nutritional approaches to countering the effects of viral respiratory infections including SARS-CVoV-2 through promoting metabolic and immune fitness with probiotics and plant bioactives. Nutrients 2020; 8:921
9. Schuijt T J, et al. The gut microbiota plays a protective role in the host defense against pneumococcal pneumonia. Gut. 2016; 65:575-83
10. Haak B W, et al. Impact of gut colonization with butyrate-producing microbiota on respiratory viral infection following allo-HCT. Blood. 2018; 131:2978-298.
11. Coppola S, et al. The Protective Role of Butyrate against Obesity and Obesity-Related Diseases. Molecules. 2021; 26:682
12. Berni Canani R, et al. Potential beneficial effects of butyrate in intestinal and extraintestinal diseases. World J Gastroenterol. 2011; 17:1519-1528
13. Russo R, et al. In vivo bioavailability and in vitro toxicological evaluation of the new butyric acid releaser N-(1-carbamoyl-2-phenyl-ethyl) butyramide. Biomed Pharmacother. 2021; 137:111385
14. Bousquet J, et alNrf2-interacting nutrients and COVID-19: time for research to develop adaptation strategies. Clin Transl Allergy. 2020; 10:58
15. Hathaway D, et al. Omega 3 Fatty Acids and COVID-19: A Comprehensive Review. Infect Chemother. 2020; 52:478-495.
16. Wang Z Z, et al. A small molecule compound berberine as an orally active therapeutic candidate against COVID-19 and SARS: A computational and mechanistic study. FASEB J 22 Mar. 2021 https://doi.org/10.1096/fj.20201792R.
17. Kaul T N, et al. Antiviral effect of flavonoids on human viruses. J Med Virol. 1985; 15:71-79
18. Wu W, et al. Quercetin as an Antiviral Agent Inhibits Influenza A Virus (IAV) Entry. Viruses. 2015; 8:6
19. Gonzalez-Ochoa G, et al. Modulation of Rotavirus severe gastroenteritis by the combination of probiotics and prebiotics. Arch Microbiol. 2017; 199:953-961
20. Ahanchian H, Jafari S A. Probiotics and Prebiotics for Prevention of Viral Respiratory Tract Infections. Probiotics, Prebiotics, and Synbiotics. 2016; 575-583.
21. Park S W, et al. Antiviral activity and possible mode of action of ellagic acid identified in Lagerstroemia speciosa leaves toward human rhinoviruses. BMC Complement Altern Med. 2014; 14:171
22. Kamboj A. Antiviral activity of plant polyphenols. J Pharm Res. 2012; 5:2402-2412
23. Pujari R, Banerjee G. Impact of prebiotics on immune response: from the bench to the clinic. Immunol Cell Biol. 2021; 99:255-73.
24. Galmés S, et al. Current State of Evidence: Influence of Nutritional and Nutrigenetic Factors on Immunity in the COVID-19 Pandemic Framework. Nutrients. 2020; 12:2738.

What is claimed is:

1. A method for preventing or reducing SARS-CoV-2 entry in a subject's cells, said method comprising administering to the subject of an effective amount of a composition comprising an amide derivative of butyric acid selected from the group consisting of N-(1-carbamoyl-2-phenyl-ethyl)butyramide (FBA), N-(1-butyroyl-carbamoyl-2-phenyl-ethyl)butyramide, 5-benzyl-2-propyl-1H-imidazol-4(5H)-one, N-(1-oxo-3-phenyl-1-(piperidin-1-yl)propan-2-yl)butyramide, N-(1-oxo-3-phenyl-1-(pyrrolidin-1-yl)propan-2-yl)butyramide, N-(1-(methylcarbamoyl)-2-phenylethyl)butyramide, N-(1-(ethylcarbamoyl)-2-phenylethyl)butyramide, N-(1-(propylcarbamoyl)-2-phenylethyl)butyramide, N-(1-(butylcarbamoyl)-2-phenylethyl)butyramide, N-(1-(pentylcarbamoyl)-2-phenylethyl)butyramide, N-(1-carbamoyl-2-phenylethyl)-N-methylbutyramide, N-(1-carbamoyl-2-phenylethyl)-N-ethylbutyramide, N-(1-carbamoyl-2-phenylethyl)-N-propylbutyramide, pharmaceutically acceptable salts, diastereoisomes and enantiomers thereof, or comprising any combination of the amide derivatives of butyric acid, pharmaceutically acceptable salts, diastereoisomes and enantiomers thereof.

2. The method of claim 1, wherein the composition comprises N-(1-carbamoyl-2-phenyl-ethyl)butyramide (FBA).

3. The method of claim 2, which wherein the composition comprises the combination of N-(1-carbamoyl-2-phenyl-ethyl)butyramide (FBA) with N-(1-butyroyl-carbamoyl-2-phenyl-ethyl)butyramide and 5-benzyl-2-propyl-1H-imidazol-4(5H)-one.

4. (canceled)

5. The method of claim 1, wherein the composition downregulates expression of the cellular-mediators angiotensin-converting enzyme 2 (ACE2) and transmembrane serine protease-2 (TMPRSS2).

6. (canceled)

7. The method of claim 1, wherein the composition further comprises one or more active compounds and/or substances selected from the group consisting of flavonoids, vitamins, minerals, curcumin, berberine, genistein, lactoferrin, omega-3-polyunsaturated fatty acids, prebiotics, probiotics and radical scavengers.

8. The method of claim 1, wherein the composition is formulated as an enteral, parenteral, topical or oral preparation.

9. The method of claim 1, wherein the composition is a food supplement including one or more dietarily acceptable vehicles and/or excipients.

10. The method of claim 1, wherein the composition is a food product, a drink or a beverage.

11. The method of claim 1, wherein the composition is an oral pharmaceutical preparation including one or more pharmaceutically acceptable vehicles and/or excipients.

12. (canceled)

13. A method for preventing or reducing an inflammatory cascade induced by a SARS-CoV-2 infection in a subject, said method comprising administering to the subject of an effective amount of a composition comprising an amide derivative of butyric acid selected from the group consisting of N-(1-carbamoyl-2-phenyl-ethyl)butyramide (FBA), N-(1-butyroyl-carbamoyl-2-phenyl-ethyl)butyramide, 5-benzyl-2-propyl-1H-imidazol-4(5H)-one, N-(1-oxo-3-phenyl-1-(piperidin-1-yl)propan-2-yl)butyramide, N-(1-oxo-3-phenyl-1-(pyrrolidin-1-yl)propan-2-yl)butyramide, N-(1-(methylcarbamoyl)-2-phenylethyl)butyramide, N-(1-(ethylcarbamoyl)-2-phenylethyl)butyramide, N-(1-(propylcarbamoyl)-2-phenylethyl)butyramide, N-(1-(butylcarbamoyl)-2-phenylethyl)butyramide, N-(1-(pentylcarbamoyl)-2-phenylethyl)butyramide, N-(1-carbamoyl-2-phenylethyl)-N-methylbutyramide, N-(1-carbamoyl-2-phenylethyl)-N-ethylbutyramide, N-(1-carbamoyl-2-phenylethyl)-N-propylbutyramide, pharmaceutically acceptable salts, diastereoisomes and enantiomers thereof, or comprising any combination of the amide derivatives of butyric acid, pharmaceutically acceptable salts, diastereoisomes and enantiomers thereof.

14. The method of claim 13, wherein the composition comprises N-(1-carbamoyl-2-phenyl-ethyl)butyramide (FBA).

15. The method of claim 14, wherein the composition comprises the combination of N-(1-carbamoyl-2-phenyl-ethyl)butyramide (FBA) with N-(1-butyroyl-carbamoyl-2-phenyl-ethyl)butyramide and 5-benzyl-2-propyl-1H-imidazol-4(5H)-one.

16. The method of claim 13, wherein the composition downregulates expression of the pro-inflammatory cytokines interleukin (IL)-15, monocyte chemoattractant protein-1 (MCP-1) and tumor necrosis factor-alpha (TNF-α).

17. The method of claim 13, wherein the composition further comprises one or more active compounds and/or substances selected from the group consisting of flavonoids, vitamins, minerals, curcumin, berberine, genistein, lactoferrin, omega-3-polyunsaturated fatty acids, prebiotics, probiotics and radical scavengers.

18. The method of claim 13, wherein the composition is formulated as an enteral, parenteral, topical or oral preparation.

19. The method of claim 13, wherein the composition is a food supplement including one or more dietarily acceptable vehicles and/or excipients.

20. The method of claim 13, wherein the composition is a food product, a drink or a beverage.

21. The method of claim 13, wherein the composition is an oral pharmaceutical preparation including one or more pharmaceutically acceptable vehicles and/or excipients.
